(19) United States
(12) Patent Application Publication
(10) Pub. No.: US 2023/0181027 A1
(43) Pub. Date: Jun. 15, 2023

(54) METHODS AND MATERIALS FOR TREATING NEUROMYELITIS OPTICA SPECTRUM DISEASES

(71) Applicant: Mayo Foundation for Medical Education and Research, Rochester, MN (US)

(72) Inventors: Long-Jun Wu, Rochester, MN (US); Tingjun Chen, Rochester, MN (US); Vanda A. Lennon, Rochester, MN (US)

(21) Appl. No.: 17/614,133

(22) PCT Filed: May 27, 2020
(86) PCT No.: PCT/US2020/034641

§ 371 Date: Nov. 24, 2021

Related U.S. Application Data
(60) Provisional application No. 62/868,053, filed on Jun. 28, 2019.

(51) Int. Cl. A61B 3/024; A61K 39/395

(52) U.S. Cl. CPC A61B 3/024; A61K 39/39533

(57) ABSTRACT

This document relates to methods and materials for treating a neuromyelitis optica (NMO) spectrum disorder such as NMO. For example, one or more tetracycline antibiotics can be administered to a mammal having, or at risk of developing, a NMO spectrum disorder to treat the mammal.

### CROSS-REFERENCE TO RELATED APPLICATIONS

This application claims the benefit of U.S. Provisional Application Serial No. 62/868,053, filed Jun. 28, 2019. The disclosure of the prior application is considered part of (and is incorporated by reference in) the disclosure of this application.

### BACKGROUND

### 1. Technical Field

This document relates to methods and materials for treating a neuromyelitis optica (NMO) spectrum disorder such as NMO. For example, one or more tetracycline antibiotics can be administered to a mammal having, or at risk of developing, a NMO spectrum disorder to treat the mammal.

### 2. Background Information

NMO is a debilitating and sometimes fatal neurological autoimmune condition characterized by preferential demyelination of optic nerves and the spinal cord. While NMO symptoms can be mitigated, this disease has no cure and eventually all patients experience impairments. Thus, there is unmet medical need to identify novel and specific therapeutics for the treatment of NMO attacks and the prevention of NMO relapses. An IgG autoantibody specific for the astrocytic AQP4 water channel (NMO-IgG or AQP4-IgG) is the primary pathogenic effector of NMO (Lennon et al., Lancet 364:2106-2112 (2004); Lennon et al., J. Exp. Med. 202:473 (2005); and Roemer et al., Brain 130:1194-1205 (2007)). AQP4 is highly concentrated at astrocyte end-feet which embrace capillaries, glutamatergic synapses, nodes of Ranvier, ventricle walls and pia-glial interfaces (Szu et al., Front. Integr. Neurosci. 10:8 (2016); Hinson et al., Proc. Natl. Acad. Sci. USA 109: 1245-1250 (2012); Guo et al., Acta Neuropathol. 133:597-612 (2017); and Misu et al., Brain 130:1224-1234 (2007)). NMO-IgG mainly targets astrocytic AQP4 (Hinson et al., Proc. Natl. Acad. Sci. USA 114:5491-5496 (2017); Hinson et al., Neurology 69:2221-2231 (2007); and Hinson et al., Arch. Neurol. 66:1164-1167 (2009)).

### SUMMARY

This document provides methods and materials related to treating a mammal having, or at risk of developing, a NMO spectrum disorder such as NMO. For example, this document provides methods and materials for using one or more tetracycline antibiotics (e.g., minocycline) to treat a mammal having, or at risk of developing, a NMO spectrum disorder such as NMO. In some cases, a mammal having, or at risk of developing, a NMO spectrum disorder can be administered a composition including one or more tetracycline antibiotics to treat the mammal.

Current treatments for NMO, include intravenous corticosteroid (such as methylprednisolone), plasma exchange, and other antibody-depleting therapies. These treatments can be non-specific and can induce adverse complications (Scott et al., Neurology 77:2128-2134 (2011); and Drozdowicz et al., Mayo Clin. Proc. 89:817-834 (2014)). As demonstrated herein, astrocyte-microglia interaction drives pathogenesis of NMO, and minocycline can reverse NMO-IgG (e.g., AQP4-IgG) induced motor dysfunction and can reduce NMO-IgG induced microglia-astrocyte interactions. The identification of a previously unrealized role for microglia in NMO pathogenesis, provides a unique target for treating mammals having, or at risk of developing, NMO. For example, a mammal having, or at risk of developing, NMO can be treated by administering minocycline to reduce or eliminate microglia activation.

In general, one aspect of this document features methods for treating a mammal having a NMO spectrum disorder. The methods can include, or consist essentially of, administering a composition including a tetracycline antibiotic to a mammal having a NMO spectrum disorder to reduce or eliminate a motor function impairment in the mammal. The method can include identifying the mammal as being in need of a treatment for the NMO spectrum disorder. The mammal can be a human. The NMO spectrum disorder can be NMO. The motor function impairment can be decreased visual acuity, visual field defects, loss of color vision, muscle weakness, reduced sensation, perverted sensation, loss of bladder control, loss of bowel control, paraparesis, quadriparesis, neuroinflammation, vomiting, hiccups, bladder dysfunction, bowel dysfunction, confusion, seizures, coma, respiratory failure, or cognitive impairment. The tetracycline antibiotic can target microglia in the mammal. The tetracycline antibiotic can target C3a receptor (C3aR) polypeptides on the microglia. The tetracycline antibiotic can be minocycline. The composition can include from about 50 µg to about 300 µg of the minocycline.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Diagram showing the role of microglia in NMO-IgG-induced pathology. (1) On entering the central nervous system (CNS), NMO-IgG binds to AQP4 which is highly expressed on astrocyte end-feet embracing blood vessels. (2) The binding of IgG to AQP4 activates the astrocyte and causes AQP4 to be internalized. (3) Activated astrocyte releases complement C3. (4) C3 cleavage yields C3a fragment. C3a receptors are highly expressed on microglia. (5) Microglia respond to astrocytic signaling and physically interact with the astrocyte. (6) Myelin damage ensues.

FIG. 2. NMO-IgG intrathecal infusion induces motor impairment. (a) Timeline of drug treatments and intrathecal infusion surgery. (b) Rotarod tests reveal that infusion of NMO-IgG, but not control-IgG, induces dose-dependent motor dysfunction (measured by fall latency). (c) Representative images of gait before and after NMO-IgG or control IgG infusion (stride length of painted paws). (d) Stride length measurement shows significant functional impairment of gait in NMO-IgG recipient mice when compared with control-IgG recipients. (e) Immunofluorescence staining confirms AQP4 protein presence in the spinal cord of WT mice (upper right) but not in AQP4 knock-out (KO) mice (lower right). (f) Rotarod analysis shows NMO-IgG infusion fails to induce motor dysfunction in AQP4 KO mice (measured by fall latency on Rotarod). (g) Infusion of AQP4-specific monoclonal mouse IgG induces the same motor impairment phenotype as NMO-IgG. (h) Rotarod analysis shows infusion of AQP4-specific monoclonal mouse IgG fails to induce motor dysfunction in AQP4 KO mice (measured by latency to fall). Data represent mean ± SEM in behavior tests, n=5 for NMO-IgG and n=4 for control-IgG groups, ***P<0.001, Two-way ANOVA.

FIG. 3. NMO-IgG induces loss of AQP4, astrocyte activation, and demyelination. (a) Representative longitudinal spinal cord images show AQP4 (upper) and DAPI (lower) staining after 5 days of NMO-IgG infusion. Scale bar, 1 mm. (b) Western blot shows that NMO-IgG significantly reduces AQP4 protein in the spinal cord. (c) Representative transverse section images show staining of AQP4 (green) and endothelium (CD31, red) at L4 level of spinal cord at day 5 after infusing control IgG (upper) or NMO-IgG (lower). Scale bar, 20 µm (left), (d) Quantification of the length of AQP4 covered endothelium (CD31) in L4 spinal cord. (e) Representative images show astrocytic cytoplasm (GFAP) staining at Day 5 after infusing NMO-IgG (upper, longitudinal and lower right, transverse section L4 spinal cord) and control IgG (lower left, transverse section L4 spinal cord). Scale bar, 1 mm, upper and 200 µm, lower. (f) Quantification of astrocyte (GFAP⁺) cell numbers and volume after 5 days of infusing control IgG and NMO-IgG. (g) Luxol fast blue staining at L4 level of spinal cord shows myelin retention at day 5 of control IgG infusion (upper) and myelin loss after NMO-IgG infusion (lower). (h) Representative transverse section images show NeuN staining (left) and eclipse picture of NeuN⁺ neurons (right) at L4 level of spinal cord at day 5 after infusing control IgG (upper) or NMO-IgG (lower). Scale bar, 200 µm. (i) Bar graph depicts NeuN+ neuron numbers at L4 level of spinal cord of each group. Scale bar, 200 µm. Data represent mean ± SEM, in immunostaining, n = 20 sections (from 5 animals), in Western blot, n = 4. ***P<0.001, t-test.

FIG. 4. Microglial activation in response to NMO-IgG infusion. (a) Representative images of longitudinal (upper) and transverse L4 spinal cord sections (lower) show Iba1 staining after NMO-IgG infusion. Scale bar, 1 mm, upper, and 200 µm, lower. (b) Quantification of Ibal⁺cell numbers after 5 days’ infusion of NMO-IgG or control IgG. (c) Representative raw (green) and transformed skeletal (gray) images show microglia morphology after infusion for 3 and 5 days with control IgG or NMO-IgG. (d) Quantification of microglial process length and branch number at day 5 after control IgG or at day 3 or day 5 after NMO-IgG infusion. Sholl analysis of microglia at day 5 after control IgG or NMO-IgG infusion. (e) CD68 staining in CX3CR1^{GFP} mice at L4 level of spinal cord at day 5 of control IgG (upper) or NMO-IgG (lower) infusion. Scale bar, 20 µm, and quantification of CD68⁺ /CX3CR1^{GFP} cell number at day 5 after control IgG or NMO-IgG infusion. Data represent mean ± SEM, n = 20 sections (from 5 animals). ***P<0.001, *P<0.05, t-test.

FIG. 5. Microglial activation (C1q upregulation) in response to NMO-IgG infusion. (a) CX3CR1^{GFP} cells at day 5 of control-IgG infusion, and at days 3 and 5 of NMO-IgG infusion. Scale bar, 200 µm. (b) Dual staining reveals complement C1q immunoreactivity (red) and Iba1(green)-positive cells in L4 spinal cord of representative mice on day 5 of IgG infusion. Few C1q+ cells were observed in control-IgG recipients but C1q was robustly expressed in enlarged Iba1 + microglia (arrow heads) of NMO-IgG recipient mice. Bar graph quantifies C1q+ microglia in both IgG recipient groups (ImageJ). Scale bar, 20 µm. Data represent mean ± SEM (4 sections from 5 individual animals). ***P<0.001, t-test.

FIG. 6. Microglial activation in NMO-IgG recipient mice is AQP4-dependent. Lysosomal CD68 (red) was upregulated by NMO-IgG in wild-type (AQP4^{+/+}) mice, but not in AQP4-null mice, nor by control-IgG. L4 spinal cord, day 5 of IgG infusion. Scale bar, 20 µm.

FIG. 7. Microglial ablation prevents NMO-IgG-induced motor impairment. (a) Timeline of tamoxifen and diphtheria toxin injections and tissue harvesting for immunostaining. Bar graph shows Ibal⁺cell numbers at day 1, day 3, day 5 and day 7 after DT administration. (b) Representative image shows Iba1staining in L4 spinal cord at each time point of microglia ablation. (c) Timeline of microglia ablation in relation to NMO-IgG infusion. Rotarod testing shows that ablation of microglia prior to infusing NMO-IgG prevents motor dysfunction (latency to fall). (d) Representative transverse section images of L4 spinal cord show NeuN staining (upper) and eclipse rendering of NeuN⁺ neurons (lower) after 5 days of NMO-IgG infusion, with microglia ablated (left) or intact (right). Scale bar, 200 µm. Bar graph depicts NeuN+ cell numbers (L4 spinal cord) in each group. (e) Luxol fast blue staining (L4 spinal cord) shows myelin retention with (left) or without (right) microglia ablation at day 5 after NMO-IgG infusion. Scale bar, 100 µm. (f) Representative transverse section images of L4 spinal cord show AQP4 (red) and CD31+ endothelium (red) after 5 days NMO-IgG infusion in the absence and presence of microglia. Scale bar, 20 µm (left), (d) Quantification of the length of AQP4 covered CD31 in L4 spinal cord. Data represent mean ± SEM, in behavior test, n=5 for NMO-IgG group or n=4 for control IgG group, ***P<0.001, Two-way ANOVA, in immunostaining, n = 20 sections each group.

FIG. 8. NMO-IgG induces microglia-astrocyte interactions. (a) Representative L4 spinal cord images show microglia (Ibal+) and astrocytes (GFAP+). Both cell types are enlarged with overlapping processes in NMO-IgG recipients but not in control IgG recipients. Bar graph shows overlap area quantified by ImageJ software. (b) Venn diagram shows the percentage of overlap area of each cell quantified by ImageJ software. (c) Representative images showing enhanced interaction events (i.e., overlap) of genetically labelled CX3CR1+ microglia [green] and GFAP-immunoreactive astrocytes [red]) in NMO-IgG recipients but not in control IgG group. (d) Number of interaction events increased from 10.6 ± 3.8 to 141.8 ± 24.2. (e) Representative in vivo (2 photon) images show microglia (labeled by CX3CR1^{GFP}) and astrocyte (labeled by SR101). Before NMO-IgG intrathecal injection, few microglial processes were observed (upper); after NMO-IgG injection, microglial processes extended and converged around astrocytes (lower). (f) Quantification of microglia process density surrounding astrocyte before/after NMO-IgG injection. Data represent mean ± SEM, n = 20 sections (from each of 5 mice). For in vivo imaging, n=3. ***P<0.001, t-test.

FIG. 9. NMO-IgG initiates astrocyte-microglia interactions. Interaction of CD1 1b+ microglia (green) and GFAP+ astrocytes (red) is inferred from enlargement of those cells and overlapping of their processes in dual immune-stained L4 spinal cord of NMO-IgG-recipient mice compared with control-IgG recipients. Bar graph shows overlap quantified by ImageJ software. Data represent mean ± SEM, n = 20 sections (from each of 5 mice), ***P<0.001, t-test.

FIG. 10. Complement signaling in microglia-astrocyte interaction and NMO pathology. (a) Representative figures show complement C3 and GFAP double staining in astrocytes of L4 spinal cord in an NMO-IgG recipient, and quantification of C3⁺ astrocyte at day 5 after NMO-IgG or control IgG infusion. (b) Representative image of C3a receptor and Iba1double staining shows C3a receptor is specifically expressed by microglia. (c) In vitro imaging shows process of microglia convergent to C3a, and quantification of microglial processes surrounding C3a. (d) C3 and C3aR staining in spinal cord tissue but absent in C3 KO or C3aR KO mice respectively. (e) In both C3 KO and C3aR KO mice, NMO-IgG infusion fails to induce motor dysfunction measured by Rotarod test. (f) Representative figures from C3 KO and C3aR KO mice; NMO-IgG infusion does not induce microglia-astrocyte interaction. (g) Quantification of microglia-astrocyte interaction events at day 5 of NMO-IgG or control IgG infusion in C3 KO and C3aR KO mice. Data represent mean ± SEM, for immunostaining, n = 20 sections (from each of 5 mice), in behavior test, n = 5 animals in each group. ***P<0.001, Two-way ANOVA.

FIG. 11. Genetic ablation of microglia did not prevent NMO-IgG upregulation of astrocytic C3. Dual staining reveals complement C3 (green) and GFAP (red) immunoreactivities in astrocytes of L4 spinal cord of mice at day 5 of NMO-IgG infusion, with (upper) or without (lower) microglia ablation. Bar graph quantifies C3+ astrocytes in both ablation and non-ablation groups after NMO-IgG infusion. Data represent mean ± SEM, n = 20 sections (from each of 5 mice).

FIG. 12. AQP4 loss after NMO-IgG infusion does not require complement C3 signaling. Astrocytic AQP4 (green) and endothelial CD31 (red) in spinal cord of IgG recipients, control (upper) and NMO (lower) in C3-null (left) and C3aR-null (right) mice. Scale bar, 20 µm.

FIG. 13. Microglia inhibitor minocycline reverses NMO-IgG-induced motor dysfunction and microglia-astrocyte interaction. (a) Intrathecally injected minocycline (150 µg/day) prevented the NMO-IgG-induced motor dysfunction in rotarod test. (b and c) Minocycline reduced microglia-astrocyte interaction. (d) NMO-IgG upregulation of astrocyte complement C3 is not altered by minocycline co-infusion. (e) Intrathecal injection of minocycline after 2 days of NMO-IgG infusion reverses the motor impairment. Data represent mean ± SEM, for immunostaining, n = 20 sections (from each of 5 mice), ***P<0.001, t-test, in behavior test, n = 5 animals in each group. ***P<0.001, Two-way ANOVA.

FIG. 14. CSF1 receptor inhibitor PLX3397 depletes microglia and suppresses NMO-IgG-induced motor dysfunction. (a) Timeline of PLX3397 treatment in NMO model (upper) and Iba1 immunofluorescence in L4 spinal cord at Day 5 after NMO-IgG infusion under PLX3397 chow or control chow (b) Bar graph shows in at Day 5 after NMO-IgG infusion, Iba1 ⁺ cell density in PLX3397 chow treated group is significantly lower than control chow treated group. (c) Rotarod test show PLX3397 chow treated animals’ motor function are significantly better than control chow treated animals. Data represent mean ± SEM, for immunostaining, n = 9 sections (from each of 3 mice), ***P<0.001, t-test, in behavior test, n = 3 animals in each group. ***P<0.001, Two-way ANOVA.

### DETAILED DESCRIPTION

This document provides methods and materials related to treating a mammal (e.g., a human) having, or at risk of developing, a NMO spectrum disorder such as NMO. For example, this document provides methods and materials for using one or more tetracycline antibiotics (e.g., minocycline) to treat a mammal having, or at risk of developing, a NMO spectrum disorder such as NMO. In some cases, a mammal having, or at risk of developing, a NMO spectrum disorder can be administered a composition including one or more tetracycline antibiotics to treat the mammal.

In some cases, a mammal (e.g., a human) having, or at risk of developing, a NMO spectrum disorder such as NMO can be administered one or more tetracycline antibiotics (e.g., minocycline) to reduce or eliminate one or more NMO spectrum disorder impairments (e.g., NMO-IgG induced impairments) and/or one or more symptoms of a NMO spectrum disorder. For example, one or more tetracycline antibiotics (e.g., minocycline) can be administered to a mammal (e.g., a human) as described herein to reduce the severity of one or more NMO spectrum disorder impairments and/or one or more symptoms of a NMO spectrum disorder by, for example, 10, 20, 30, 40, 50, 60, 70, 80, 90, 95, or more percent. NMO spectrum disorder impairments can be vision impairments and/or motor function impairments. Examples of NMO spectrum disorder impairments and symptoms of a NMO spectrum disorder include, without limitation, decreased visual acuity, visual field defects, loss of color vision, muscle weakness, reduced sensation, perverted sensation, loss of bladder control, loss of bowel control, paraparesis, quadriparesis, neuroinflammation, vomiting, hiccups, bladder dysfunction, bowel dysfunction, confusion, seizures, coma, respiratory failure, and cognitive impairment.

In some cases, a mammal (e.g., a human) having, or at risk of developing, a NMO spectrum disorder such as NMO can be administered one or more tetracycline antibiotics (e.g., minocycline) to reduce or eliminate one or more NMO spectrum disorder pathologies (e.g., NMO-IgG induced pathologies). For example, one or more tetracycline antibiotics (e.g., minocycline) can be administered to a mammal (e.g., a human) as described herein to reduce the severity of one or more NMO spectrum disorder pathologies by, for example, 10, 20, 30, 40, 50, 60, 70, 80, 90, 95, or more percent. Examples of NMO spectrum disorder pathologies include, without limitation, inflammatory demyelination, inflammatory lesions (e.g., with or without demyelination), inflammatory cell invasions, and blood-brain barrier damage.

Any appropriate mammal having, or at risk of developing, a NMO spectrum disorder can be treated as described herein (e.g., by administering one or more tetracycline antibiotics). Examples of mammals having, or at risk of developing, a NMO spectrum disorder that can be treated as described herein include, without limitation, humans, non-human primates (e.g., monkeys), dogs, cats, horses, cows, pigs, sheep, mice, rat, and rabbit. In some cases, a human having, or at risk of developing, a NMO spectrum disorder can be treated by administering one or more tetracycline antibiotics to the human.

Any appropriate NMO spectrum disorder can be treated as described herein (e.g., by administering one or more tetracycline antibiotics). In some cases, a NMO spectrum disorder can be a NMO spectrum disorder accompanied by the presence of anti-AQP4 autoantibodies (e.g., NMO-IgG or AQP4-IgG). In some cases, a NMO spectrum disorder can be a NMO spectrum disorder accompanied by the presence of anti-myelin oligodendrocyte glycoprotein (MOG) autoantibodies (e.g., MOG-IgG). In some cases, a NMO spectrum disorder can be a NMO spectrum disorder can lack autoantibodies. Examples of NMO spectrum disorders that can be treated as described herein can include, without limitation, AQP4-IgG-positive NMO (also referred to as Devic’s disease), limited forms of Devic’s disease (e.g., single events of longitudinally extensive myelitis, recurrent events of longitudinally extensive myelitis, bilateral simultaneous optic neuritis, and bilateral recurrent optic neuritis), Asian optic-spinal MS, longitudinally extensive myelitis, optic neuritis (e.g., optic neuritis associated with systemic autoimmune disease), myelitis associated with lesions in the brain (e.g., in specific brain areas such as the hypothalamus, periventricular nucleus, and brainstem), and seronegative NMO (e.g., NMO lacking an autoantibody). In some cases, a mammal (e.g., a human) having, or at risk of developing, NMO can be treated by administering one or more tetracycline antibiotics to the mammal.

In some cases, methods for treating a mammal (e.g., a human) having, or at risk of developing, a NMO spectrum disorder, also can include identifying a mammal as having, or as being at risk of developing, a NMO spectrum disorder. Any appropriate method can be used to identify a mammal as having, or as being at risk of developing, a NMO spectrum disorder. For example, neurological examinations (e.g., neurological examinations for muscle strength, coordination, sensation, cognitive functions such as memory and thinking, and vision and speech), neurological imaging (e.g., magnetic resonance imaging (MRI) to detect lesions or damaged areas of the brain, optic nerves and spinal cord), blood tests (e.g., blood tests looking for the presence of autoantibodies such as NMO-IgG (e.g., AQP4-IgG)), lumbar punctures (e.g., to test the amounts and types of leukocytes, proteins, and/or antibodies in the spinal fluid), stimuli response tests (e.g., to learn how well the brain responds to stimuli such as sounds, sights, touch, and/or memory) can be used to identify a mammal as having, or as being at risk of developing, a NMO spectrum disorder.

Once identified as having, or as being at risk of developing, a NMO spectrum disorder, a mammal (e.g., a human) can be administered, or instructed to self-administer, one or more tetracycline antibiotics. A tetracycline antibiotic can be any appropriate tetracycline antibiotic (e.g., any antibiotic in the tetracycline family of antibiotics). As used herein, a tetracycline antibiotic has a linear fused tetracyclic nucleus (rings designated A, B, C and D) to which a variety of functional groups (designated as R groups; e.g., chloride, methyl, and hydroxyl groups) are attached as shown below.

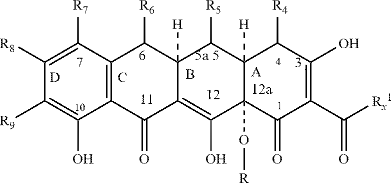

In some cases, a tetracycline antibiotic can be a broad-spectrum tetracycline antibiotic. In some cases, a tetracycline antibiotic can be a second-generation tetracycline antibiotic. In some cases, a tetracycline antibiotic can target (e.g., can selectively target) microglia (e.g., microglial C3aR polypeptides). For example, a tetracycline antibiotic can selectively target C3aR polypeptides on microglia to reduce or eliminate microglia activation. In some cases, a tetracycline antibiotic can cross the blood-brain barrier. Examples of tetracycline antibiotics that can be used to treat a mammal having, or at risk of developing, a NMO spectrum disorder as described herein include, without limitation, minocycline, tetracycline-3, and doxycycline. In some cases, a tetracycline antibiotic that can be used as described herein can be minocycline. A chemical formula for a minocycline can be as follows.

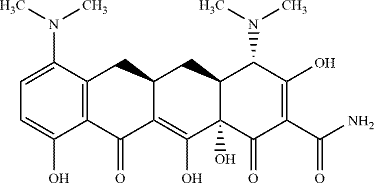 [SMILES: CN(C)c1ccc(O)c2c1C[C@H]1C[C@H]3[C@H](N(C)C)C(O)=C(C(N)=O)C(=O)[C@@]3(O)C(O)=C1C2=O]

For example, a mammal (e.g., a human) having, or at risk of developing, a NMO spectrum disorder such as NMO can be treated by administering minocycline to the mammal. In some cases, tetracycline antibiotics that can be used to treat a mammal having, or at risk of developing, a NMO spectrum disorder as described herein include, without limitation, those described in Robert et al., Nat. Neurosci. 18: 1081-1083 (2015); Sharma et al., Circ Res. 124:727-736 (2019); and Sultan et al., Front. Neurosci. 7:31 (2013).

In some cases, methods for treating a mammal (e.g., a human) having, or at risk of developing, a NMO spectrum disorder, can include administering to the mammal one or more agents that can deplete microglia. Examples of agents that can be used as described herein to deplete microglia include, without limitation, plexxikon compounds (see, e.g., Elmore et al., Neuron 82:380-397 (2014)).

In some cases, one or more tetracycline antibiotics (e.g., minocycline) can be formulated into a composition (e.g., a pharmaceutically acceptable composition) for administration to a mammal having, or at risk of developing, a NMO spectrum disorder such as NMO. For example, one or more tetracycline antibiotics can be formulated together with one or more pharmaceutically acceptable carriers (additives) and/or diluents. Pharmaceutically acceptable carriers, fillers, and vehicles that can be used in a pharmaceutical composition described herein include, without limitation, saline, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol (PEG; e.g., PEG400), sodium carboxymethylcellulose, polyacrylates, waxes, polyethylene-polyoxypropylene-block polymers, wool fat, and corn oil.

In some cases, when a composition containing one or more tetracycline antibiotics (e.g., minocycline) is administered to a mammal having, or at risk of developing, a NMO spectrum disorder such as NMO, the composition can be designed for oral or parenteral (including, without limitation, subcutaneous, intramuscular, intravenous, intradermal, intra-cerebral, intrathecal, or intraperitoneal (i.p.) injection) administration to the mammal. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition containing one or more tetracycline antibiotics (e.g., minocycline) can be administered to a mammal having, or at risk of developing, a NMO spectrum disorder such as NMO in any appropriate amount (e.g., any appropriate dose). Effective amounts can vary depending on the route of administration, the age and general health condition of the subject, excipient usage, the possibility of co-usage with other therapeutic treatments such as use of other agents, and the judgment of the treating physician. An effective amount of a composition containing one or more tetracycline antibiotics can be any amount that can treat a mammal having, or at risk of developing, a NMO spectrum disorder such as NMO without producing significant toxicity to the mammal. For example, an effective amount of minocycline can be from about 50 micrograms (µg) to about 300 µg (e.g., from about 50 µg to about 250 µg, from about 50 µg to about 200 µg, from about 50 µg to about 150 µg, from about 50 µg to about 100 µg, from about 100 µg to about 300 µg, from about 150 µg to about 300 µg, from about 200 µg to about 300 µg, from about 250 µg to about 300 µg, from about 100 µg to about 250 µg, from about 150 µg to about 200 µg, or from about 100 µg to about 200 µg) per day. The effective amount can remain constant or can be adjusted as a sliding scale or variable dose depending on the mammal’s response to treatment. Various factors can influence the actual effective amount used for a particular application. For example, the frequency of administration, duration of treatment, use of multiple treatment agents, route of administration, and severity of the NMO spectrum disorder in the mammal being treated may require an increase or decrease in the actual effective amount administered.

A composition containing one or more tetracycline antibiotics (e.g., minocycline) can be administered to a mammal having, or at risk of developing, a NMO spectrum disorder such as NMO in any appropriate frequency. The frequency of administration can be any frequency that can treat a mammal having, or at risk of developing, a NMO spectrum disorder without producing significant toxicity to the mammal. For example, the frequency of administration can be from about once a week to about once a month, from about twice a month to about once a month, or from about once a day to about once a week. The frequency of administration can remain constant or can be variable during the duration of treatment. As with the effective amount, various factors can influence the actual frequency of administration used for a particular application. For example, the effective amount, duration of treatment, use of multiple treatment agents, and route of administration may require an increase or decrease in administration frequency.

A composition containing one or more tetracycline antibiotics (e.g., minocycline) can be administered to a mammal having, or at risk of developing, a NMO spectrum disorder such as NMO for any appropriate duration. An effective duration for administering or using a composition containing one or more tetracycline antibiotics can be any duration that can treat a mammal having, or at risk of developing, a NMO spectrum disorder without producing significant toxicity to the mammal. For example, the effective duration can vary from several months to several years or to a lifetime. In some cases, the effective duration can range in duration from about 10 years to about a lifetime. Multiple factors can influence the actual effective duration used for a particular treatment. For example, an effective duration can vary with the frequency of administration, effective amount, use of multiple treatment agents, and route of administration.

In some cases, methods for treating a mammal (e.g., a human) having, or at risk of developing, a NMO spectrum disorder, can include administering to the mammal one or more tetracycline antibiotics (e.g., minocycline) as the sole active ingredient to treat the mammal. For example, a composition containing one or more tetracycline antibiotics can include the one or more tetracycline antibiotics as the sole active ingredient in the composition that is effective to treat a mammal having, or at risk of developing, a NMO spectrum disorder.

In some cases, methods for treating a mammal (e.g., a human) having, or at risk of developing, a NMO spectrum disorder, can include administering to the mammal one or more tetracycline antibiotics (e.g., minocycline) and also administering to the mammal one or more (e.g., one, two, three, four, five or more) additional treatments that are effective against one or more NMO spectrum disorder impairments and/or one or more symptoms of a NMO spectrum disorder to treat the mammal. Examples of treatments for one or more NMO spectrum disorder impairments and/or one or more symptoms of a NMO spectrum disorder in a mammal include, without limitation, administering to the mammal one or more active agents (e.g., therapeutic agents) that are effective against one or more NMO spectrum disorder impairments and/or one or more symptoms of a NMO spectrum disorder such as immunosuppressants (e.g., azathioprine, mycophenolate mofetil, mitoxantrone, intravenous immunoglobulin (IVIG), and cyclophosphamide), corticosteroids (e.g., methylprednisolone, and prednisone), agents that deplete B cells (e.g., rituximab), immunomodulators such as agents that neutralize or deplete complement components (e.g., anti-C5 antibodies such as eculizumab), subjecting the mammal to plasmapheresis, and/or subjecting the mammal to hematopoietic stem cell transplantation (HSCT). In some cases, the treatments for one or more NMO spectrum disorder impairments and/or one or more symptoms of a NMO spectrum disorder can be performed together with the administration of the one or more tetracycline antibiotics (e.g., minocycline). For example, a composition containing one or more tetracycline antibiotics also can include one or more additional active agents that are effective against one or more NMO spectrum disorder impairments and/or one or more symptoms of a NMO spectrum disorder. In some cases, the one or more treatments for one or more NMO spectrum disorder impairments and/or one or more symptoms of a NMO spectrum disorder can be performed independent of the administration of the one or more tetracycline antibiotics (e.g., minocycline). When the one or more treatments for one or more symptoms of a NMO spectrum disorder are performed independent of the administration of the one or more tetracycline antibiotics, the one or more tetracycline antibiotics can be administered first, and the one or more treatments for one or more symptoms of a NMO spectrum disorder performed second, or vice versa.

In certain instances, a course of treatment can be monitored. In some cases, methods described herein also can include monitoring the severity or progression of a NMO spectrum disorder such as NMO in a mammal. Any appropriate method can be used to monitor the severity or progression of a NMO spectrum disorder in a mammal. In some cases, one or more NMO spectrum disorder impairments (e.g., NMO-IgG induced impairments) can be assessed using any appropriate methods and/or techniques, and can be assessed at different time points. For example, physical examinations (e.g., eye examinations and/or motor function testing) can be used to assess NMO spectrum disorder vision impairments (e.g., decreased visual acuity, visual field defects, and loss of color vision). For example, ambulation status, coordination analysis, and/or gait analysis can be used to assess NMO spectrum disorder motor function impairments (e.g., muscle weakness, reduced sensation, perverted sensation, loss of bladder control, loss of bowel control, paraparesis, and quadriparesis). In some cases, one or more NMO spectrum disorder pathologies (e.g., NMO-IgG induced pathologies such as inflammatory demyelination, inflammatory lesions (e.g., with or without demyelination), inflammatory cell invasions, blood-brain barrier damage, loss of AQP4, and/or loss of one or more glutamate transporters (e.g., EAAT2 and/or EAAT1)) can be assessed using any appropriate methods and/or techniques, and can be assessed at different time points. For example, laboratory tests, imaging techniques, and/or biopsies can be used to assess NMO spectrum disorder pathologies (e.g., inflammatory demyelination, inflammatory lesions (e.g., with or without demyelination), inflammatory cell invasions, blood-brain barrier damage, loss of AQP4, and/or loss of one or more glutamate transporters (e.g., EAAT2 and/or EAAT1)). In some cases, one or more symptoms of a NMO spectrum disorder such as NMO can be assessed using any appropriate methods and/or techniques, and can be assessed at different time points. For example, neurological examinations (e.g., neurological examinations for muscle strength, coordination, sensation, cognitive functions such as memory and thinking, and vision and speech), neurological imaging (e.g., magnetic resonance imaging (MRI) to detect lesions or damaged areas the brain, optic nerves and spinal cord), blood tests (e.g., blood tests looking for the presence of autoantibodies such as NMO-IgG (e.g., AQP4-IgG)), lumbar punctures (e.g., to test the levels of immune cells, proteins, and/or antibodies in the spinal fluid), stimuli response tests (e.g., to learn how well the brain responds to stimuli such as sounds, sights, touch, and/or memory) can be used to assess one or more symptoms of a NMO spectrum disorder (e.g., neuroinflammation, vomiting, hiccups, bladder dysfunction, bowel dysfunction, confusion, seizures, coma, respiratory failure, and cognitive impairment).

The invention will be further described in the following examples, which do not limit the scope of the invention described in the claims.

### EXAMPLES

### Example 1: Astrocyte-Microglia Interaction Drives Pathogenesis of Neuromyelitis Optica

To investigate what signals drive microglial activation in NMO and how microglia may participate in the pathology, an informative in vivo murine model of NMO was developed that utilizes chronic intrathecal infusion of NMO patient-derived or monoclonal AQP4-specific IgGs. Using microglial depletion approaches combined with genetic knockouts, it was found that microglia participate in NMO pathophysiology by interacting with astrocytes in a complement C3 dependent manner. These results reveal unexpected complement-mediated astrocyte-microglia crosstalk in NMO pathogenesis, which can be targeted for therapeutic interceptions.

### Results

### Development of a Murine Model of NMO With Evolving Motor Function Impairments

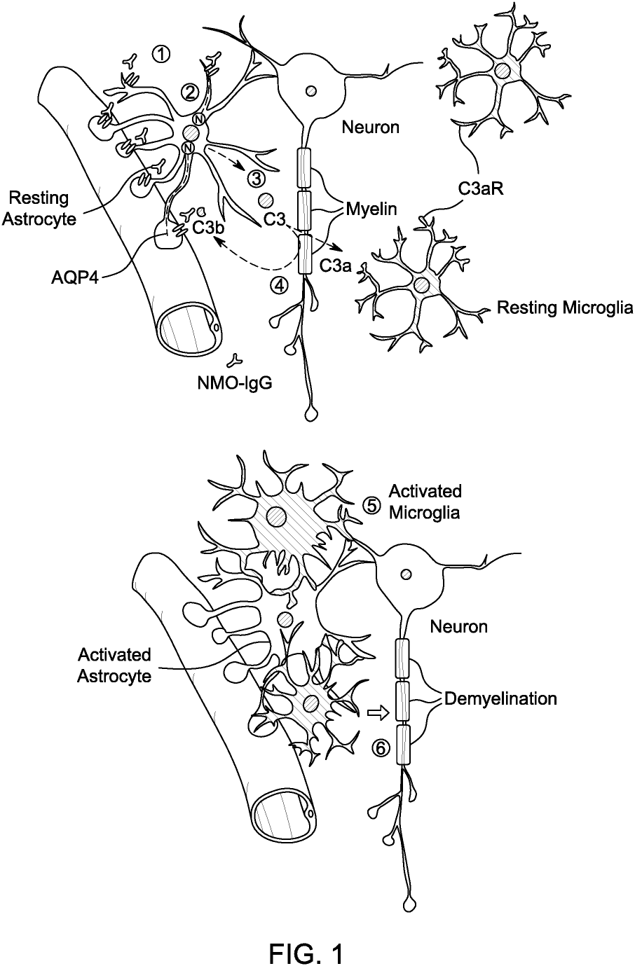

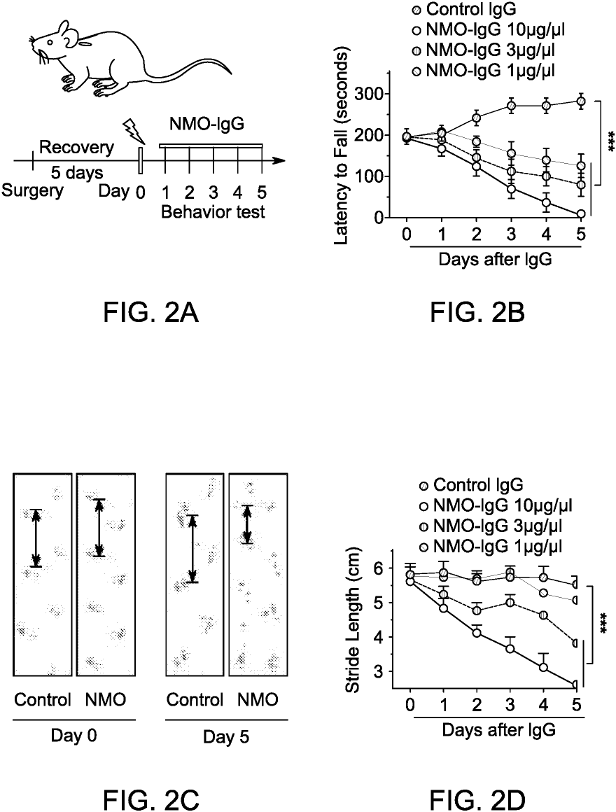

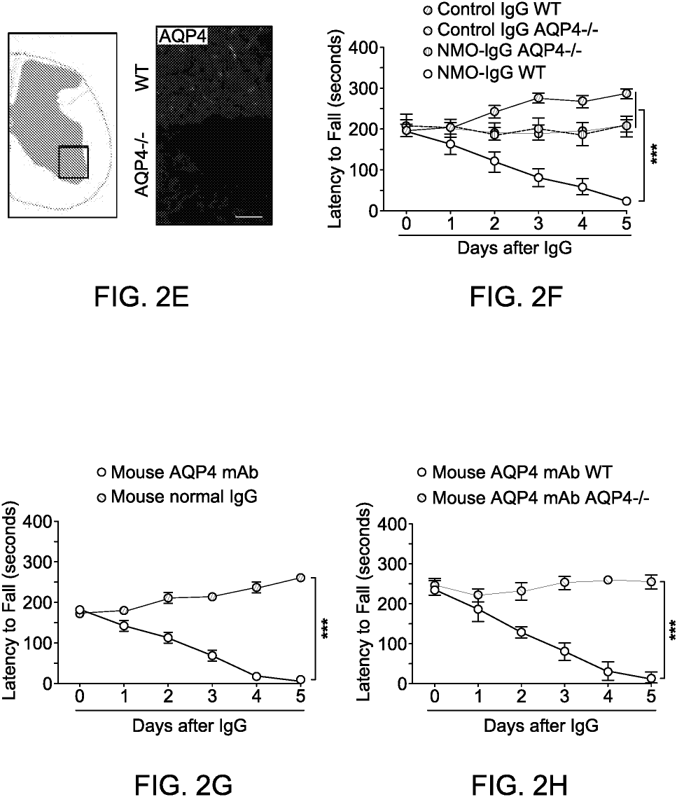

To investigate the cellular and molecular mechanisms underlying NMO pathology, a murine model was developed that utilizes IgG purified from NMO patient serum (NMO-IgG) or monoclonal AQP4-specific IgG. IgG collected from healthy individuals, or from healthy control subjects (control-IgG), or a control monoclonal IgG of irrelevant specificity was used in each experiment. NMO-IgG and control-IgG was purified by protein G adsorption (FIG. 1). An osmotic mini-pump delivery system loaded with either NMO-IgG, AQP4-IgG or control-IgG was placed subcutaneously and IgG was infused continuously into the spinal subarachnoid space via intrathecal catheter for 5 to 7 days (FIG. 2A). The development of significant motor deficits and paralysis was observed in the hind limbs over the course of treatment with NMO-IgG or AQP4-IgG, but not control-IgG. To quantify the loss of motor function, rotarod analysis was used and it was found that NMO-IgG induced severe, dose-dependent, and evolving motor impairments evidenced by shorter latency to fall (FIG. 2B). On day 5 of infusion with either 10 µg/µL, 3 µg/µL, or 1 µg/µL NMO-IgG, mice displayed average latency to fall of 4, 93, and 131 seconds respectively, while an average of 190 seconds was observed prior to NMO-IgG infusion. In contrast, mice infused with control-IgG did not display motor deficits when compared to baseline, but instead gradually improved rotarod performance suggesting motor learning (FIG. 2B). Gait analysis was also performed using ink tracking as described elsewhere (see, e.g., Zhang et al., J. Autoimmun. 53:67-77 (2014)). It was found that NMO-IgG, but not control-IgG, gradually reduced stride length. After 5 days of either 10 g/µL, 3 µg/µL, or 1 µg/µL NMO-IgG treatment, stride lengths decreased from an average of 5.7 cm to 2.6, 3.8, and 5.0 cm respectively (FIGS. 2C and 2D).

To determine AQP4 involvement in our NMO model, motor dysfunction following NMO-IgG infusion was compared in wild-type (WT) mice and AQP4 knockout (AQP4^{-/-}) mice. Deficiency of AQP4 was verified via post-mortem CNS tissue immunostaining (FIG. 2E). It was found that rotarod performance of AQP4^{-/-}mice after NMO-IgG infusion was indistinguishable from that of mice receiving control IgG. By contrast, WT mice infused with NMO-IgG displayed motor dysfunction (FIG. 2F). Further, a mouse monoclonal IgG specific for an extracellular epitope of mouse AQP4 induced similar motor dysfunction in wild type mice but not in AQP4-null mouse (FIGS. 2G, H). Together, these results demonstrate that astrocytic AQP4 is the likely target for patient-derived AQP4-IgG to initiate the evolving motor dysfunction in this murine model of NMO.

### NMO-IgG Induces Loss of AQP4, Astrocyte Activation, and Demyelination

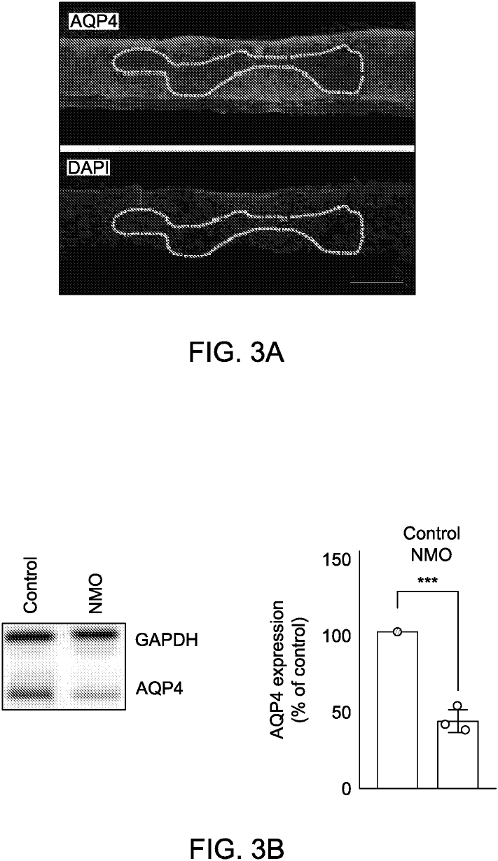

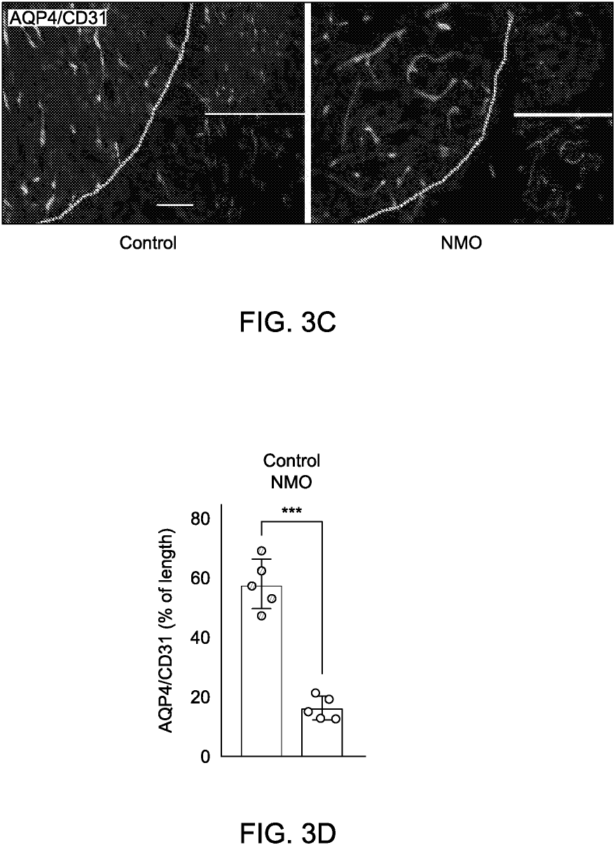

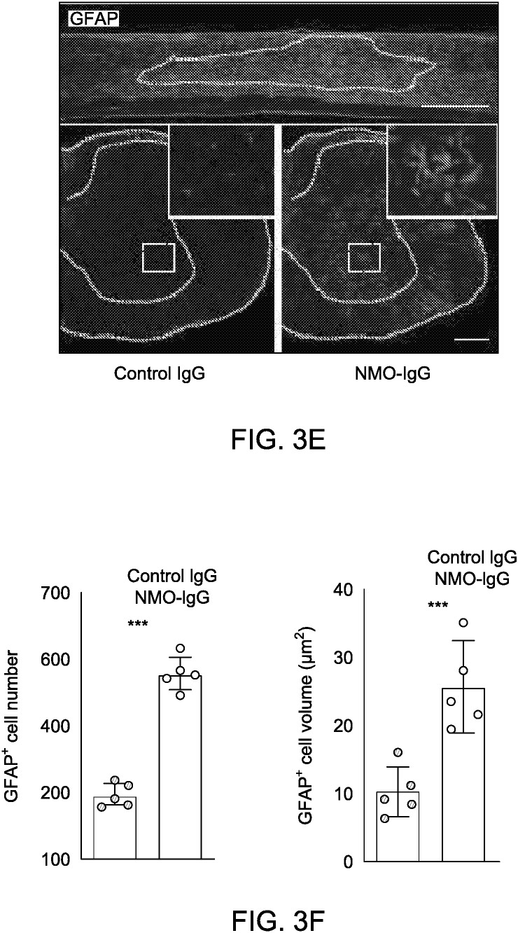

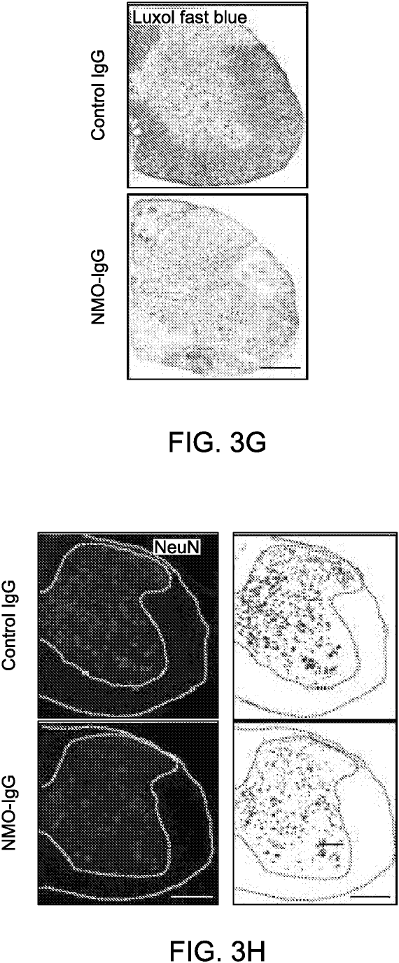

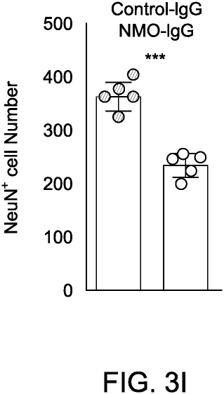

AQP4 immunostaining was performed on both longitudinal and transverse spinal cord sections from mice infused with NMO-IgG. At day 5 of infusion, spinal cord AQP4 immunoreactivity was significantly reduced, particularly in the region surrounding NMO-IgG infusion (FIG. 3A). Western blot results confirmed significant loss of AQP4 in spinal cord tissue of NMO-IgG recipient mice compared with control-IgG recipients (FIG. 3B). The co-localization of AQP4 with the vasculature marker CD31 indicated the typical location of AQP4 expression in astrocytic end-feet. It was found that NMO-IgG infusion induced dramatic loss of AQP4 but left vasculature intact (FIG. 3C). Coverage of CD31⁺ vasculature by AQP4+ end-feed decreased from 62% to 19% (FIG. 3D). To determine if reduced AQP4 staining is due to loss of astrocytes, astrocytic marker GFAP was examined after NMO-IgG infusion. A significant increase in GFAP expression was found in the dorsal horn and ventral horn of spinal cord on day 5 of NMO-IgG infusion (FIG. 3E). Specifically, GFAP⁺ cell body numbers increased from 188 ± 35 to 536 ± 72 in L4 spinal cord slice (FIG. 3F). Additionally, astrocytes exhibited increased cell body volume and thicker processes in NMO-IgG treated mice when compared to control-IgG treated mice, and average area of GFAP⁺ cells increased from 7.38 ± 0.76 µm² to 32.33 ± 2.15 µm² (FIG. 3F). These results indicate that our murine model of NMO induces activation of spinal astrocytes while downregulating AQP4.

Immunostaining was performed using the neuronal marker NeuN. Significant loss of NeuN staining was observed in both dorsal and ventral horns of NMO-IgG recipient mice when compared to control-IgG recipients (FIG. 3G). Next, spinal cord myelin integrity was assessed using fast blue staining. Fast blue staining was significantly reduced in NMO-IgG recipient mice but not in control IgG recipients (FIG. 3H). Therefore, the murine NMO model displays loss of myelin and neuronal markers, characteristics found in progressive NMO lesions.

### Microglia are Activated in the Murine Model of NMO

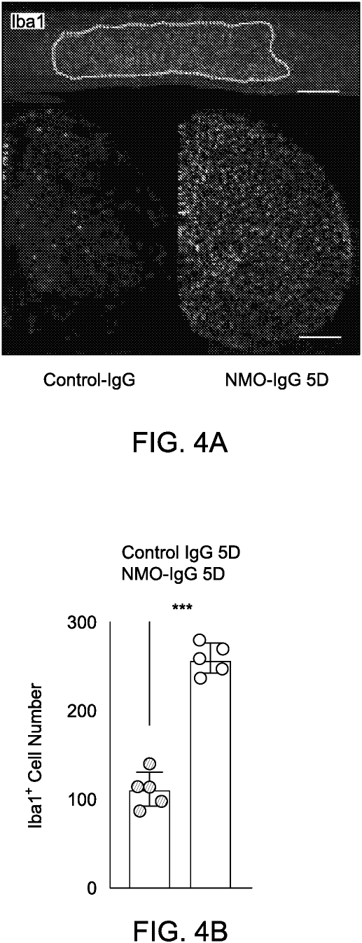

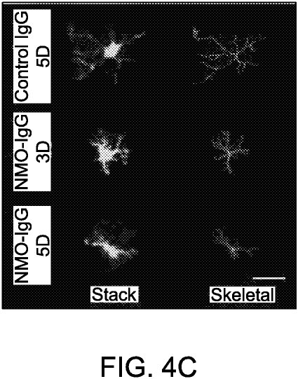

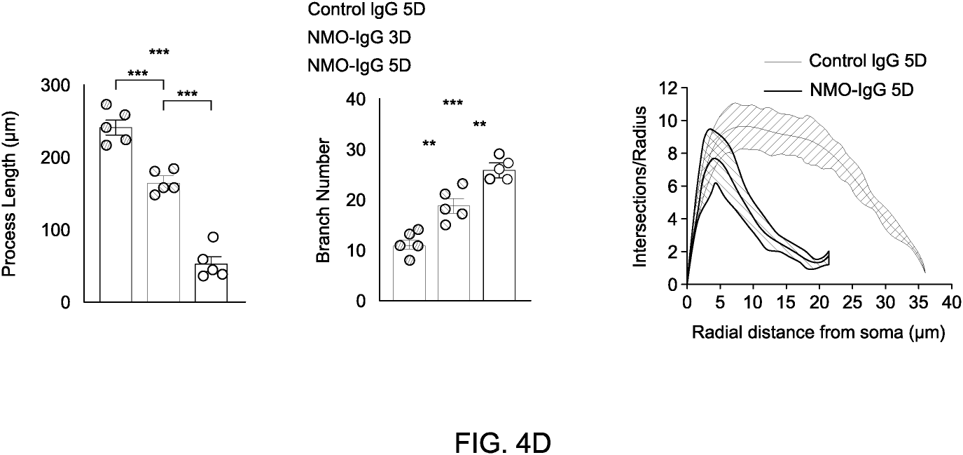

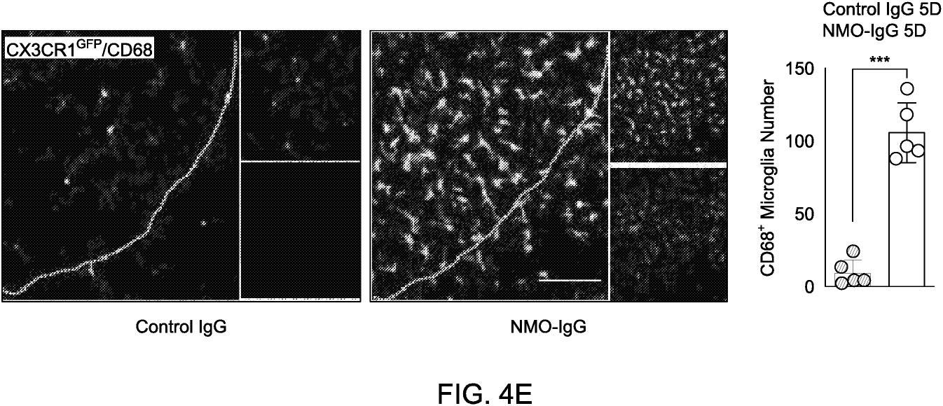

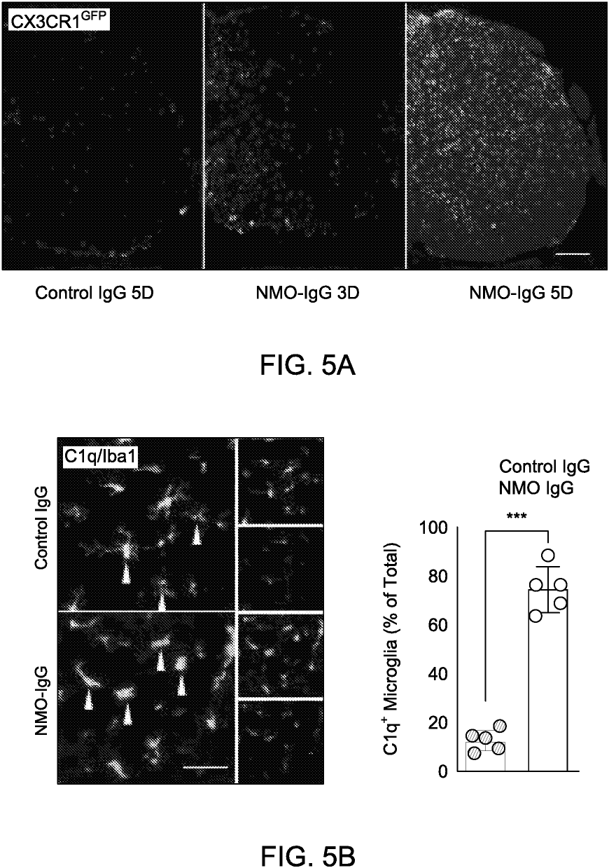

To examine microglial activation in our murine model of NMO, genetic labeling of microglia (CX3CR1^{GFP/+}) and immunostaining for the microglial marker Iba1 were used. Dramatic microglial activation was found in NMO-IgG recipient mice when compared with controls (FIG. 4A). Specifically, the intensity of Iba1 immunoreactivity, the number of Iba1⁺ microglia, and the volume of Iba1⁺ cells were all significantly increased in L4 spinal cord sections of NMO-IgG recipient mice compared with control recipients (FIGS. 4A and 4B). in CX3CR1^{GFP/+} mice, a striking increase in numbers of GFP⁺ microglia was noted after NMO IgG infusion (FIG. 5A). In addition, NMO-IgG induced striking morphological transition of microglia, from “resting” ramified to “activated” bushy phenotype in time-dependent manner (FIG. 4C). Sholl analysis demonstrated further that microglia indeed had less complex processes after NMO-IgG exposure (FIG. 4D).

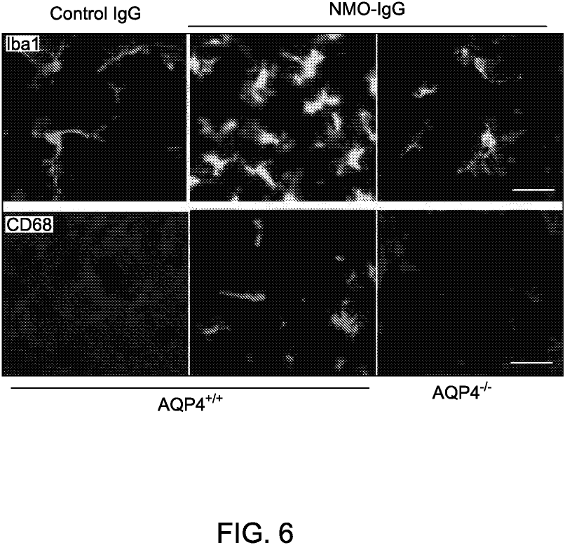

The expression of CD68, a marker for microglial activation, was examined and it was largely increased in microglia following NMO-IgG exposure (FIG. 4E). Moreover, complement component C1q, another microglial activation marker, was also increased in the cytoplasm of microglia after NMO-IgG infusion compared with control IgG recipients (FIG. 5B). Finally, in AQP4-null mice, microglial activation as determined by Iba1⁺ cell number and CD68 expression, was not altered by NMO-IgG exposure when compared to controls (FIG. 6). Together, these results indicate that induction of microglial activation and astrocytic and neuronal pathology by NMO-IgG is presumably secondary to its binding to astrocytic AQP4.

### Microglial Ablation Prevents NMO-IgG Induced Motor Dysfunction

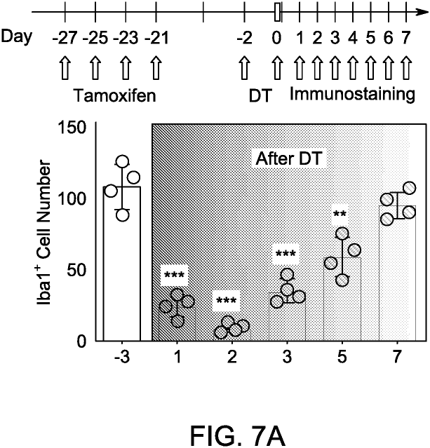

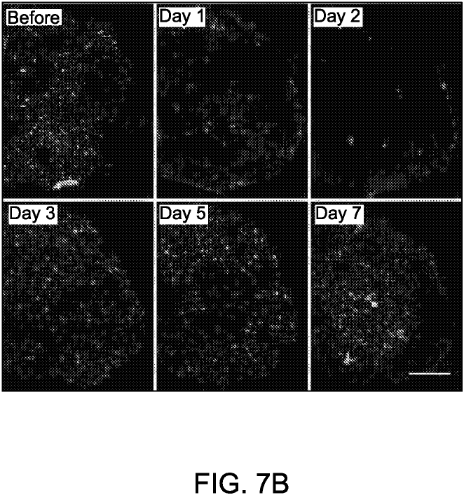

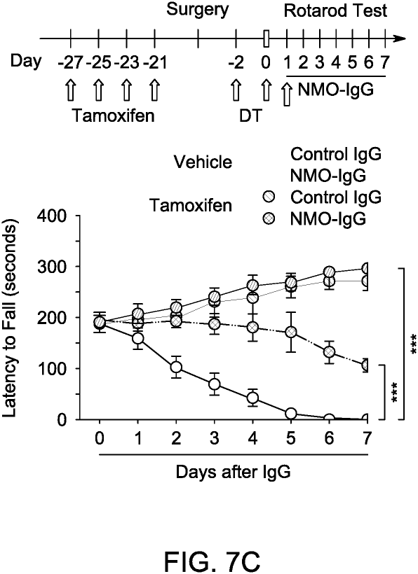

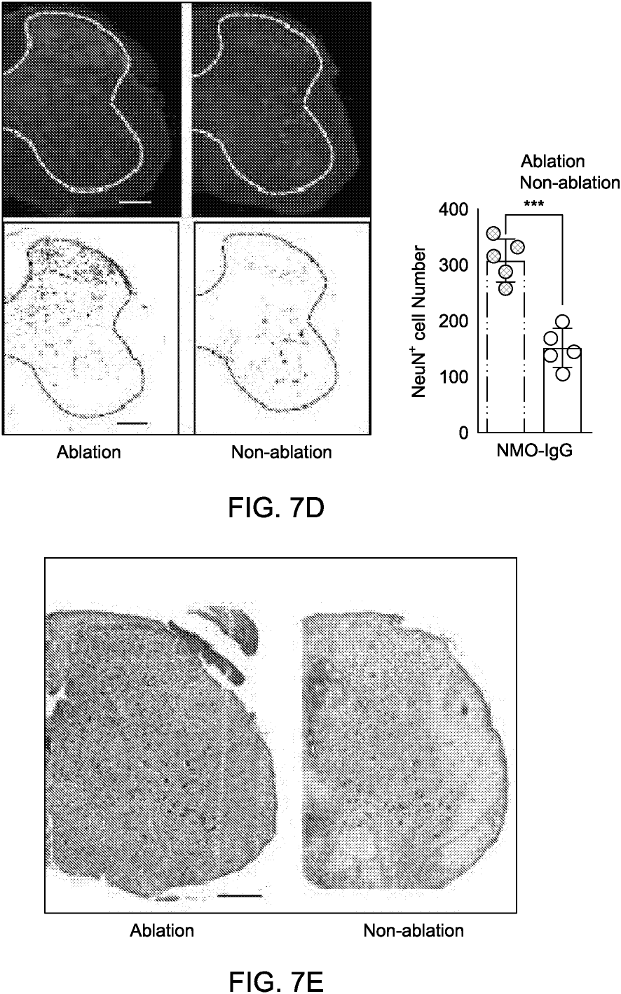

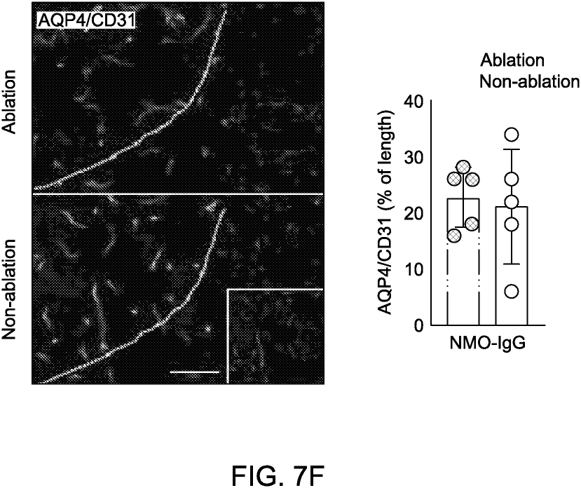

Microglial activation after NMO-IgG infusion indicates a potential role for microglia in NMO pathogenesis. To test this idea, microglia ablation approaches were utilized to directly examine the requirement of microglia in NMO-induced motor deficits. This was accomplished by using CX3CR1^{CreER/+}: R26^{iDTR/+} mice, which are induced by tamoxifen treatment (150 mg/Kg, i.p.) to express diphtheria toxin receptor (DTR) in microglia. Spinal microglia were mostly depleted 1 to 3 days after administering diphtheria toxin (DT, 50 µg/Kg, i.p.) and these microglia gradually repopulated 5 to 7 days after DT (FIGS. 7A and 7B).

To test the effects of microglia depletion and repopulation on NMO-IgG induced motor dysfunction DT was injected to deplete microglia prior to NMO-IgG infusion. It was found that microglial ablation strongly suppressed the onset of motor dysfunction in the mouse NMO model (FIG. 7C). Moreover, when microglial repopulation occurred approximately 5 days post-DT, severe motor dysfunction emerged (FIG. 7C). Further examining the pathology, it was found that after 3 days of NMO infusion, less neuronal damage, as determined by NeuN staining (FIG. 7D), and demyelination, as determined by fast blue staining (FIG. 7E), occurred in mice that underwent microglia ablation. Microglia ablation did not alter the reduced expression of AQP4 or vascular coverage by AQP4 after NMO-IgG infusion (FIG. 7F). Together, these observations suggest that microglia play a central role in the initiation of behavioral impairments in response to NMO-IgG.

### NMO-IgG Induces Microglia-Astrocyte Interactions

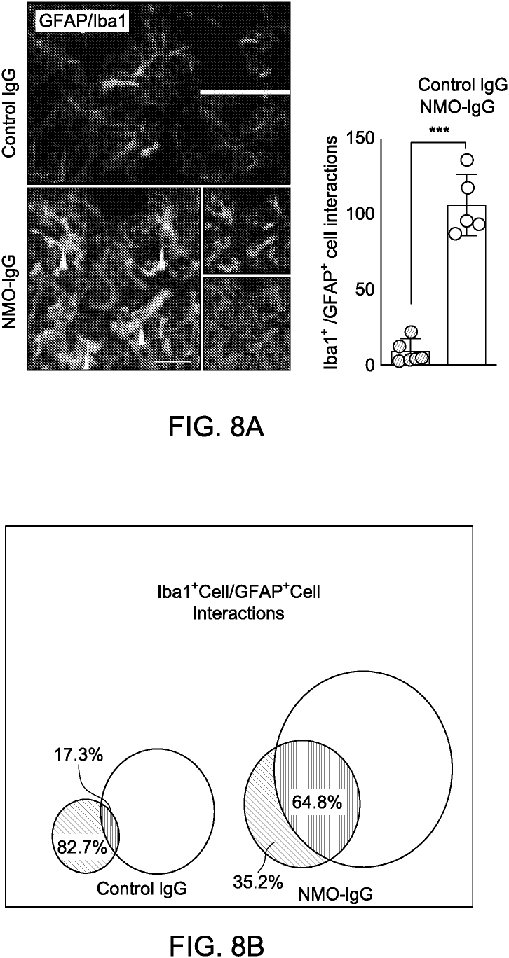

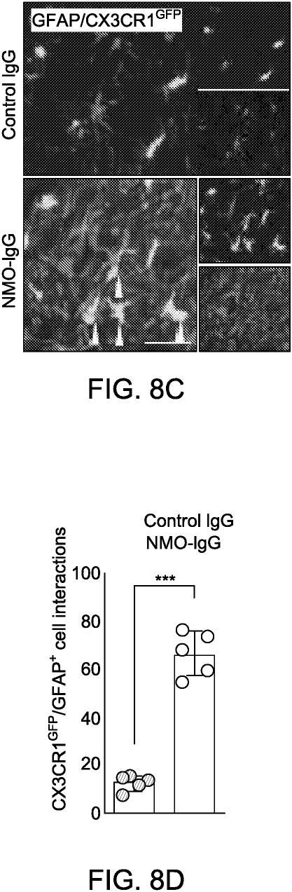

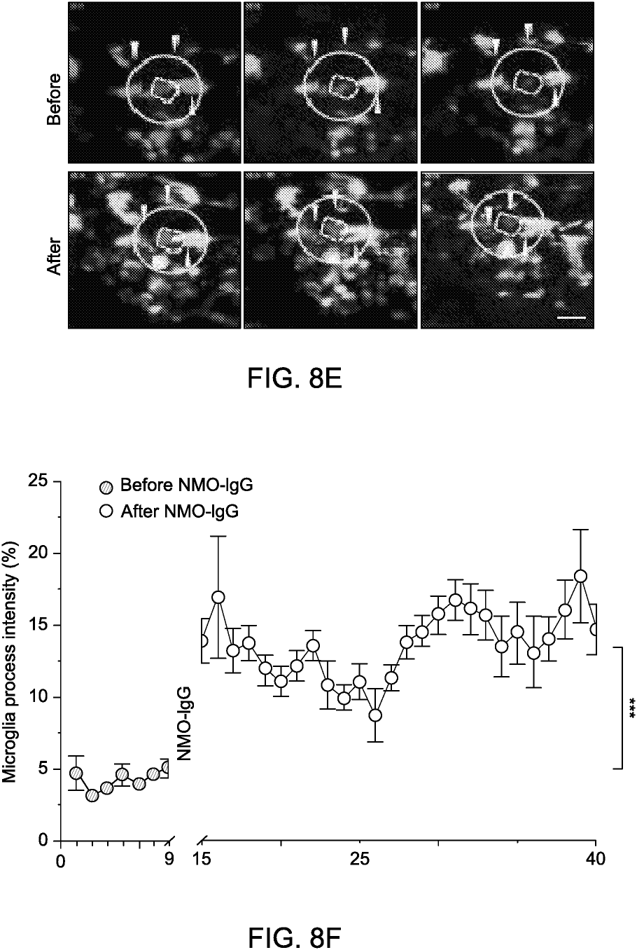

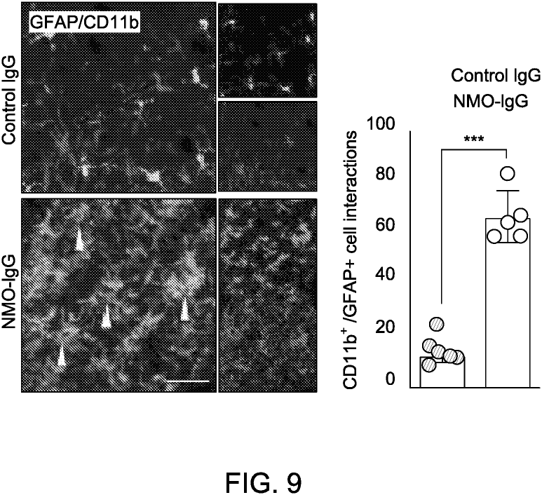

These results demonstrate a critical role for microglia in NMO pathogenesis. However, the question remains as to what cellular and molecular mechanisms underlie microglial function in NMO. This investigation of spinal astrocyte and microglia activation revealed an intriguing coalescence of astrocytes and microglia following NMO-IgG infusion (FIGS. 8A and 8B). Under control conditions, GFAP⁺ astrocytes rarely overlap with Iba1⁺ microglia. However, spatial overlapping was abundant after NMO-IgG exposure. Quantification of Iba1⁺/GFAP⁺ interaction revealed that astrocyte-microglia coalescence increased 10 fold (FIGS. 8A and 8B). Specifically, on average only 17% of microglia interact with astrocytes under control conditions while 65% of microglia overlap with astrocytes after NMO-IgG infusion. However, the overall number of each cell type only increased 2 to 3 fold (FIG. 8B). When CX3CR1^{GFP/+} mice were used, GFAP staining consistently revealed increased overlapping of GFP⁺ microglia and GFAP⁺ astrocytes after NMO-IgG infusion (FIGS. 8C and 8D). In addition, double staining of CD11b (microglia) and GFAP (astrocytes) also revealed increased astrocyte-microglia coalescence (FIG. 9). These data strongly indicate increased astrocyte-microglial interaction in response to NMO exposure, which is not simply attributable to increased numbers of microglia and astrocytes.

To further investigate this unusual microglia-astrocyte interaction, in vivo 2-photon imaging of microglia (labeled with CX3CR1^{GFP/+}) and astrocytes (labeled with SR101) in the spinal cord as performed. In response to NMO-IgG infusion, significant microglial process extension towards astrocytes was observed (FIG. 8D). Microglial process convergence events were observed shortly after the second injection of NMO-IgG (8 hours after the first injection, in spite of very few convergence events happened during the period between two injections (FIG. 8E). This type of dynamic and real-time interaction between astrocytes and microglia has not been documented previously in any NMO models or any other disease context. Therefore, these results demonstrate that NMO-IgG triggers direct interaction of microglia with astrocytes, which may have critical pathophysiological implications for NMO lesion evolution.

### Complement Signaling in Microglia-Astrocyte Interaction and NMO Pathology

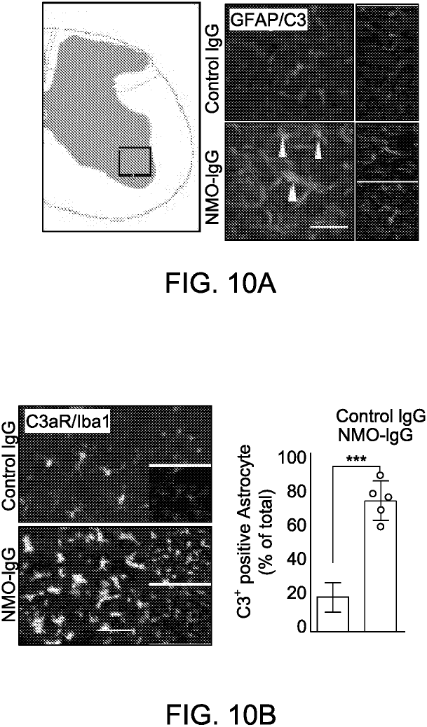

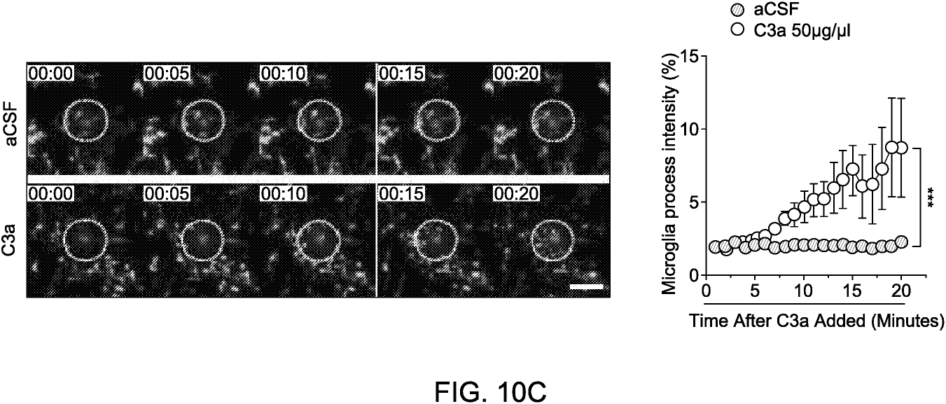

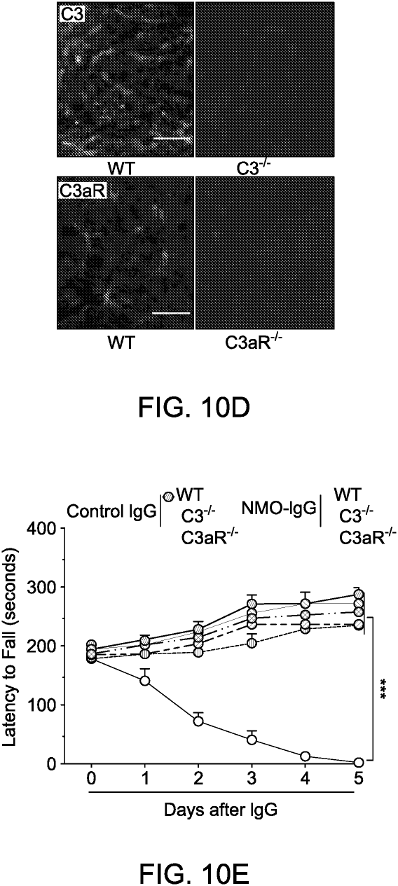

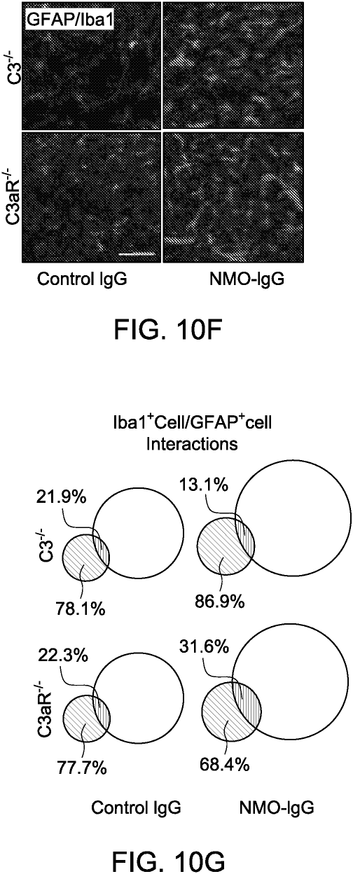

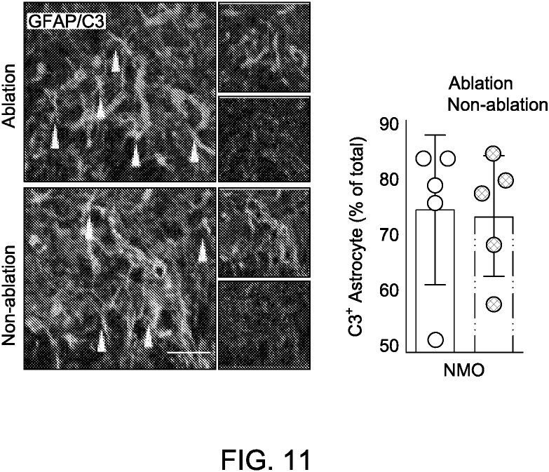

Astrocytes exposed in culture to NMO-IgG increase their production and secretion of all complement components, except C1q, which is made by activated microglia (see, Howe et al., Glia 62:692-708 (2014)). In this murine NMO model, a dramatic increase of C3 expression was observed in GFAP⁺ astrocytes following NMO-IgG exposure (FIG. 10A). In contrast, C3 expression was principally restricted to the lumen of blood vessels following control-IgG exposure. The percentage of C3⁺ astrocytes in spinal cord of mice receiving NMO-IgG was ~5-fold higher than that of control-IgG recipients (FIG. 10A). C3 upregulation in astrocytes is independent of microglia, as a similar C3 increase was found after microglia ablation (FIG. 11).

The cleavage of the complement component C3 produces two signaling molecules, C3a and C3b. Immunostaining revealed that C3a receptor (C3aR) is exclusively expressed on microglia and is upregulated after NMO-IgG infusion (FIG. 10B). Moreover, using an in situ microglial process chemoattraction assay (described elsewhere, see, e.g., Wu et al., Glia 55: 810-821 (2007); and Eyo et al., J. Neurosci. 34: 10528-10540 (2014)), it was found that C3a-containing pipettes induce microglial process chemotaxis within 30 minutes (FIG. 10C). Together, these results indicate that NMO-IgG induces C3 expression in astrocytes, which may trigger microglia-astrocyte interactions via microglial C3aR.

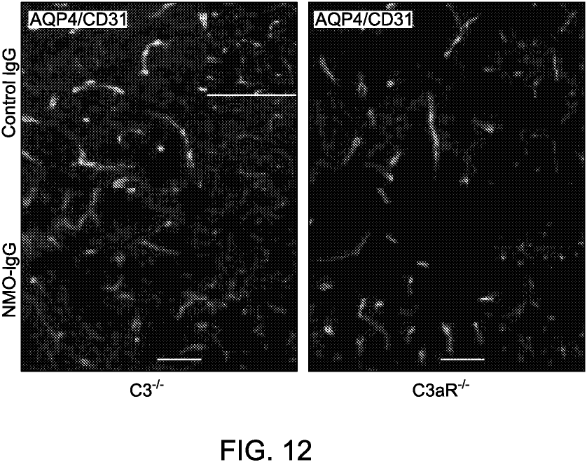

To further investigate the role of C3-dependent complement signaling in NMO-IgG-induced pathology and motor dysfunction, NMO-IgG was infused into C3^{-/-} and C3aR^{-/-} mice (FIG. 10D). Although NMO-IgG failed to induce motor function impairments in either C3^{-/-} or C3aR^{-/-} mice (FIG. 10E), AQP4 loss was evident in both strains of mice (FIG. 12). In addition, it was found that astrocyte activation was preserved in both C3^{-/-} and C3aR^{-/-} mice. However, microglia activation and microglia-astrocyte interactions were largely attenuated in these mice (FIGS. 10F and 10G). Thus, NMO-IgG triggered astrocytic activation and AQP4 loss in C3^{-/-} and C3aR^{-/-} mice, but in the absence of C3 or C3aR microglial activation, microglia-astrocyte interaction, or behavioral dysfunction were not observed. Together, these results demonstrate that complement signaling is critical for the microglia-astrocyte interaction that is required for progression of NMO pathology and motor impairment.

### Methods

### Animals

Female mice (6-8 weeks old) were used in accordance with institutional guidelines as approved by the animal care and use committee at Mayo Clinic. C57BL/6J (Charles River) and CX3CR1^{GFP/+} mice were used as wild-type animals. AQP4 null mice were as described elsewhere (Lennon et al., J. Exp. Med. 202:473 (2005)). C3 null mice (B6;129S4-C3tm1Crr/J) and C3aR null mice (C. 129S4-C3ar1tm1Cge/J) were purchased from Jackson lab. CX3CR1^{CreER-EYFP/+} mice were as described elsewhere (Christopher et al., Cell 155: 1596-1609 (2013)). These mice were crossed with R26^{iDTR/+} (bought from Jackson Laboratory) to obtain CX3CR1^{CreER/+}:R26^{iDTR/+} mice. Mice were assigned to experimental groups randomly within a litter. Experimenters were blind to drug treatments.

### IgG Delivery Surgery

A 3.5 cm polyurethane-silicone catheter (Alzet, CA) was inserted at the condylar canal to accesses the subarachnoid space at the cisterna magna and extended to lumbar level of spinal cord. Five days later an osmotic mini-pump delivery system, containing either NMO-IgG or control-IgG, was placed subcutaneously over the right shoulder. IgG was delivered continuously for 5-7 days (1-10 µg/day) (FIG. 2A).

### Microglial Ablation

TM (Sigma) was administered as a solution in corn oil (Sigma) to mice over 4 weeks of age via i.p. injection. Animals received four doses of TM (150 mg kg⁻¹, 20 mg mL⁻¹ in corn oil) in 48 hour intervals. For total CX3CR1⁺ cell ablation, two doses of DT (Sigma, Catalogue #D0564, 50 mg kg⁻¹, 2.5 mg mL⁻¹ in PBS) were given at 3 and 5 days after the last TM treatment. Mice administered with DT only (without TM) were used as control for all ablation experiments.

### Behavioral Measurements

The rotarod tests were performed using a five-lane Rotarod apparatus (Med Assocaites Inc). The rotarod speed started from 4 rounds per minute and uniformly accelerated to 40 rounds per minute over 5 minutes. Each mouse was tested for 3 times with 15 minute intervals. For gait analysis, mouse fore and hind limbs were covered by different color ink and allowed to walk freely across a narrow strip of paper. Stride length of hind limbs was reported as the mean of 5 sequential steps.

### Fluorescent Immunostaining

Mice were deeply anaesthetized with isoflurane (5% in O2) and perfused transcardially with 20 mL PBS followed by 20 mL of cold 4% paraformaldehyde (PFA) in PBS. The spinal cord was removed and post-fixed with 4% PFA for 6 hours at 4° C. Samples were then transferred to 30% sucrose in PBS overnight. Sample sections (15 mm in thickness) were prepared on gelatin-coated glass slide with a cryostat (Leica). The sections were blocked with 10% goat serum and 0.3% Triton X-100 (Sigma) in TBS buffer for 60 min, and then incubated overnight at 4° C. with primary antibody for rabbit anti-Iba1 (1:500, Abcam, 178847), rabbit anti-CD68 (1:500, Abcam, 125212), mouse anti-GFAP (1:500, CST, 3670), rabbit anti-AQP4 (1:500, Sigma, A5971), rabbit anti-C3 (1:200, Thermo, 21349), mouse anti-C3aR (1:500, hycultbiotech, 1123), or rat anti-CD31(1:500, BD, 550274). The sections were then incubated for 60 minutes at room temperature, with secondary antibodies (1:500, Alexa Fluor 594, Life Technologies or Alexa Fluor 488, Life Technologies). The sections were mounted with Fluoromount-G (SouthernBiotech) and fluorescent images were obtained with a confocal microscope (LSM510, Zeiss). Cell counting and fluorescent signal intensity was quantified using ImageJ software (National Institutes of Health, Bethesda, MD).

### Ex Vivo Hippocampal Slice Preparation and Imaging

Hippocampal tissue slices (400 mm thick) were prepared from P14-21 mice and incubated in imaging media. Microglia were visualized by GFP. For each image in the time-series, 15 z-steps spaced 2 mm apart were collected per image (30 mm total depth). Images were taken at 5 minute intervals for up to 1 hour on Scientifica 2-photon microscope with an X20 lens. Image processing and analysis was performed using NIH Image J software.

### In Vivo Spinal Cord Imaging

Image within the spinal cord was recorded by using a 2-photon microscope (Scientifica) with a Ti:sapphire laser tuned to 900 nm (Mai Tai; Spectra Physics), Microglia stained genetically (CX3CR1^{GFP/+}) and astrocytes stained by intrathecal injection of SR101 (5 µL at 25 µM). After performing laminectomy at L5 level, the spinal column was stabilized via clamps (model STS-A; Narishige) to minimize movement artefacts. Video and images are captured 100-150 µm from the surface.

### Statistical Analysis

Quantification of Iba1 cells was done with ImageJ software (NIH Image). Data were presented as mean ±SEM. Student’s t-test and Two-way ANOVA were used to establish significance. No statistical methods were used to predetermine sample sizes.

### Example 2: Microglia as a Novel Therapeutic Target for NMO Treatment

### Results

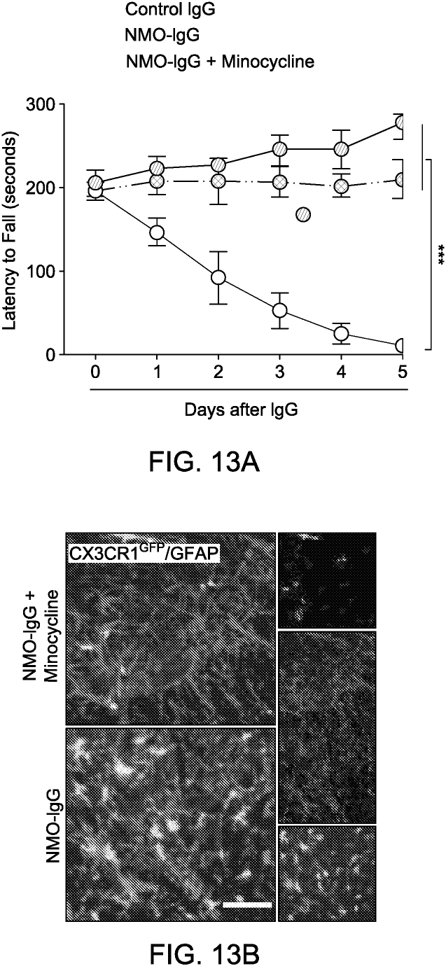

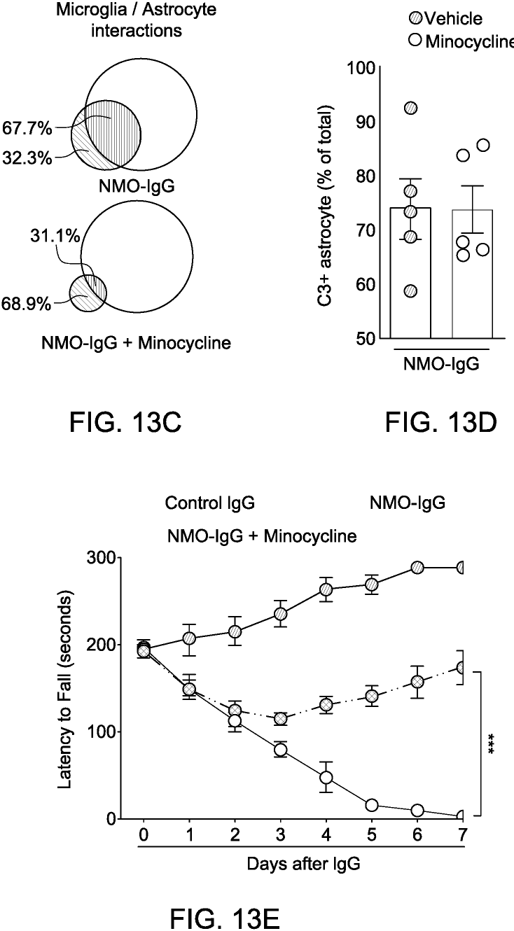

The microglia inhibitor minocycline reversed NMO-IgG induced motor dysfunction and microglia-astrocyte interaction. Intrathecally injection of minocycline (150 µg/day) prevented the NMO-IgG induced motor dysfunction in rotarod test (FIG. 13A), and reduced microglia-astrocyte interaction (FIGS. 13B and 13C). NMO-IgG upregulation of astrocyte complement C3 was not altered by minocycline (FIG. 13D). Systemic injection of minocycline after 2 days of NMO-IgG infusion reversed the motor impairment (FIG. 13E).

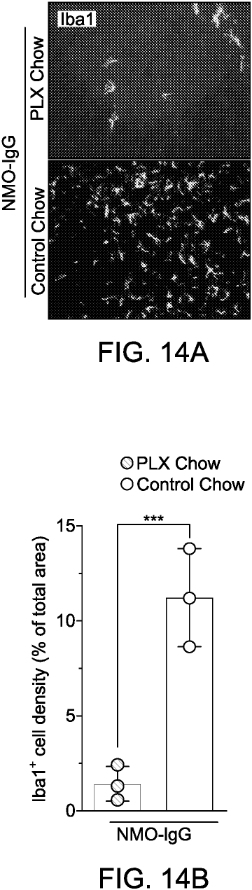

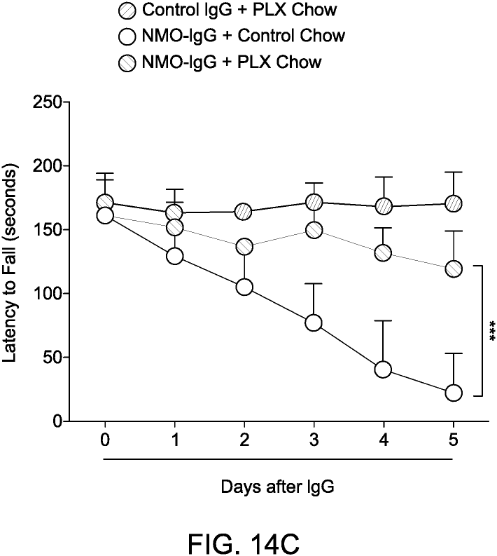

Colony stimulating factor-1 (CSF1) receptor inhibitor Pexidartinib (PLX3397) is known to deplete microglia in vivo. Animals were treated with control chow for 7 days then switched to PLX3397 chow. NMO-IgG infusion was started after 7 days of PLX3397 treatment. PLX3397 eliminated most of the microglia in the L4 spinal cord (FIGS. 14A, B). Compared with control chow-treated group, the motor function was significantly better after NMO-IgG infusion in the PLX3397-treated group (FIG. 14C).

### Methods

Minocycline hydrochloride was dissolved in PBS with 1% DMSO at 30 µg/µL and mixed with 20 µg/µL NMO-IgG to make a mixture of 15 µg/µL minocycline and 10 µg/µL NMO-IgG. 100 µL mixed drug and IgG was uploaded into osmotic pump and connected with the intrathecal infusion catheter. Pumps were implanted under the skin behind animal neck. The pump can infuse 10 µL contained liquid (150 µg minocycline and 100 µg NMO-IgG) every day. As placebo control, PBS with 1% DMSO was used instead of Minocycline.

PLX3397 was bought from Research Diets, Inc. Animals were treated with PLX3397 by feeding them PLX3397 (600 µg/mg) chow.

### Example 3: Treating a Human Having NMO

A human identified as having NMO is administered one or more tetracycline antibiotics (e.g., minocycline). After administration of one or more tetracycline antibiotics, microglia activation in the human is reduced or eliminated.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having a neuromyelitis optica (NMO) spectrum disorder, wherein said method comprises administering a tetracycline antibiotic to said mammal to reduce or eliminate a motor function impairment in said mammal.

2. The method of claim 1, wherein said method comprises identifying said mammal as being in need of a treatment for said NMO spectrum disorder.

3. The method of claim 1, wherein said mammal is a human.

4. The method of claim 1, wherein NMO spectrum disorder is NMO.

5. The method of claim 1, wherein said motor function impairment is selected from the group consisting of decreased visual acuity, visual field defects, loss of color vision, muscle weakness, reduced sensation, perverted sensation, loss of bladder control, loss of bowel control, paraparesis, quadriparesis, neuroinflammation, vomiting, hiccups, bladder dysfunction, bowel dysfunction, confusion, seizures, coma, respiratory failure, and cognitive impairment.

6. The method of claim 1, wherein said tetracycline antibiotic can target microglia in said mammal.

7. The method of claim 6, wherein said tetracycline antibiotic can target C3a receptor (C3aR) polypeptides on said microglia.

8. The method of claim 1, wherein said tetracycline antibiotic is minocycline.

9. The method of claim 8, wherein said composition comprises from about 50 µg to about 300 µg of said minocycline.
